(19) United States
(12) Patent Application Publication
(10) Pub. No.: US 2014/0005550 A1
(43) Pub. Date: Jan. 2, 2014

(54) ULTRASOUND IMAGING EQUIPMENT AND DEVICE AND METHOD FOR AUTOMATICALLY ADJUSTING USER INTERFACE LAYOUT

(71) Applicants: General Electric Company, Schenectady, NY (US); GE Medical Systems Co., LTD, Jiangsu (CN)

(72) Inventors: Yincheng Lu, Wuijiang (CN); Jiajiu Yang, Jiangsu (CN); Menachem Halmann, Bayside, WI (US); Andrew Stonefield, Fort Wayne, IN (US)

(21) Appl. No.: 13/931,002

(22) Filed: Jun. 28, 2013

(30) Foreign Application Priority Data
Jun. 29, 2012 (CN) 201210220197.0

(51) Int. Cl. A61B 8/00

(52) U.S. Cl. CPC A61B 8/465
USPC 600/459

(57) ABSTRACT

An ultrasound imaging equipment comprising a host, a probe connected to the host, a touch screen fixed to the host for providing a user interface, the user interface including a presentation area displaying images and/or various information and a control area displaying virtual buttons, and a user interface adjusting device for automatically adjusting a layout of the user interface based on a relative position between the probe and the touch screen, so as to facilitate a user's usage of the equipment.

### TECHNICAL FIELD

Embodiments of the present invention generally relate to the field of ultrasound imaging equipment, and in particular, a device and method for automatically adjusting a user interface layout for an ultrasound imaging equipment.

### BACKGROUND ART

In recent years, touch screen has been adopted as an operating interface for ultrasound imaging equipment. When using such equipment, a doctor normally is required to hold a probe in one hand to scan a patient and uses the other hand to touch various virtual buttons (or soft keys) on the touch screen to achieve some control, such as changing modes, adjusting parameters, storing images, freezing the system and the like. The locations of these virtual buttons on the screen are often fixed or need to be manually set, such that it may sometimes cause inconvenience to the doctor.

For example, in normal conditions, a hospital bed is positioned on the right side of the ultrasound equipment, and virtual buttons are displayed on the left of the touch screen, such that the doctor hold the probe in his right hand and touches virtual buttons with his left hand. However, when the ultrasound equipment is moved to a new position where a hospital bed is located on the left side of the ultrasound equipment, the doctor needs to change habits by holding the probe in his left hand and touching virtual buttons with his right hand. If the doctor wants to keep his habits, he needs to cross his hands.

However, with the left hand holding the probe and the right hand touching virtual buttons, the hand will cover the image area displayed on the right side of the virtual buttons, thereby affecting the doctor's view of diagnostic images. If some devices support manual setting of a user interface, the doctor still needs to manually change the settings of the user interface before scanning in a new position.

US Patent application No. 2009/0150814A1 filed on Dec. 6, 2007 discloses dynamic update of a user interface based on collected user interaction information, wherein various operations of the user on the user interface are recorded and analyzed to determine the user's patterns of usage (primarily the sequence of using menu options) regarding the menu on the user interface, and then to make corresponding adjustment to hierarchy of the menu options.

US Patent application No. 2009/0276726A1 filed on May 2, 2008 depicts an automated user interface adjustment scheme, wherein the user interface is adjusted responsive to an event that will result in a content being displayed outside of a viewable area of the user interface, such that the content is displayed within the viewable area of the user interface.

U.S. Pat. No. 7,620,894B1 filed on Oct. 8, 2003 provides an automatic dynamic user interface configuration scheme, wherein the user's actions are recorded and analyzed to determine the user's level of proficiency, thus configuring with corresponding menus, tool tips, help text, toolbars, etc.

U.S. Pat. No. 5,115,501 filed on Nov. 4, 1988 discloses a procedure for automatically customizing the user interface of an application, including determining a set of operations being appropriate for a user based on various relevant characteristics of the user, and presenting only the operations specified for the user in the menus, icons, application toolbars or other components of the interface.

US Patent Application No. 2007/0038088A1, filed on Aug. 4, 2006, relates to a medical imaging user interface and control scheme, including determining a relative motion between a subject patient and a probe, and selecting a motion mode for the probe to capture images of the patient if the relative motion is greater than a threshold, or selecting a stability mode for the probe to capture images of the patient if the relative motion is less than the threshold.

U.S. Pat. No. 5,119,079, filed on Sep. 17, 1990, discloses a touch screen user interface with expanding touch locations for a reprographic machine, wherein when a user's finger touches a certain zone to select a certain option, the zone is enlarged to a size accommodating the finger tip selection, and upon completing the selection, the expanded contact area is returned to the predetermined size.

Inconvenience with the use of touch screen ultrasound imaging equipment has been a long-felt problem in the prior art.

### SUMMARY OF THE INVENTION

Embodiments of the present invention provide a simple solution to the touch screen ultrasound imaging equipment, which overcomes or alleviates the aforementioned problem in the prior art and remedies the defects of the existing touch screen ultrasound imaging equipment.

According to an embodiment of the present invention, there is provided an ultrasound imaging equipment, comprising: a host; a probe connected to the host; a touch screen fixed to the host for providing a user interface, the user interface including a presentation area displaying images and/or various information and a control area displaying virtual buttons, and further comprising: a user interface adjusting device for automatically adjusting a layout of the user interface based on a relative position between the probe and the touch screen, so as to facilitate a user's usage of the equipment.

In an embodiment of the ultrasound imaging equipment of the present invention, the ultrasound imaging equipment further comprises: a state determiner for determining a state of the ultrasound imaging equipment, and for enabling or disabling the user interface adjusting device based on the determined state.

In an embodiment of the ultrasound imaging equipment of the present invention, the user interface adjusting device is disabled when the ultrasound imaging equipment is in freeze, or when the ultrasound imaging equipment is in a scan mode and the probe is in air, but is enabled when the ultrasound imaging equipment is in a scan mode and the probe is not in air.

In an embodiment of the ultrasound imaging equipment of the present invention, the user interface adjusting device comprises: a first signal transmitter and a second signal transmitter provided on the left and right sides of the touch screen respectively, for transmitting first and second signals respectively; a signal receiver disposed within the probe for receiving the first and second signals; a relative position analyzer for determining first and second distances based on the first and second signals received by the probe and for determining a relative position between the probe and the touch screen by comparing the first and second distances; and a user interface setter for automatically setting a layout of the presentation area and the control area in the user interface according to the determined relative position.

In an embodiment of the ultrasound imaging equipment of the present invention, the relative position analyzer and the user interface setter are both located within the host, and the probe transmits the received first and second signals to the relative position analyzer in a wired or wireless manner.

In an embodiment of the ultrasound imaging equipment of the present invention, the relative position analyzer is located within the probe, the user interface setter is located within the host, and the relative position analyzer transmits the determined relative position as an analytic result to the user interface setter in a wired or wireless manner.

In an embodiment of the ultrasound imaging equipment of the present invention, when the first distance is greater than the second distance, the relative position analyzer determines that the probe is located on the right side of the touch screen, and accordingly, the user interface setter sets the control area on the left side of the presentation area; when the first distance is less than the second distance, the relative position analyzer determines that the probe is located on the left side of the touch screen, and accordingly, the user interface setter sets the control area on the right side of the presentation area.

In an embodiment of the ultrasound imaging equipment of the present invention, when the absolute value of a difference between the first and second distances is less than a threshold value, the user interface setter is disabled to maintain the current user interface; when the absolute value of the difference between the first and second distances is greater than the threshold value, the user interface setter is enabled.

In an embodiment of the ultrasound imaging equipment of the present invention, the threshold value is 20 cm.

In an embodiment of the ultrasound imaging equipment of the present invention, the user interface adjusting device comprises: a signal transmitter disposed in the probe for transmitting signals; first and second signal receivers provided on the left and right sides of the touch screen respectively for receiving first and second signals from the signal transmitter respectively; a relative position analyzer for determining first and second distances based on the first and second signals and for determining a relative position between the probe and the touch screen by comparing the first and second distances; and a user interface setter for automatically setting a layout of the presentation area and the control area in the user interface according to the determined relative position.

In an embodiment of the ultrasound imaging equipment of the present invention, the relative position analyzer and the user interface setter are located within the host, and the first and second signal receivers are connected to the relative position analyzer so as to transfer the received first and second signals the relative position analyzer.

In an embodiment of the ultrasound imaging equipment of the present invention, the ultrasound imaging equipment further comprises a physical key provided on the host or a virtual key provided on the touch screen; upon pressing the key, the layout of the user interface changes.

In an embodiment of the ultrasound imaging equipment of the present invention, the ultrasound imaging equipment further comprises an acoustic control mechanism on the host, whereby when the user voices “switch the user interface”, the layout of the user interface changes.

In an embodiment of the ultrasound imaging equipment of the present invention, the ultrasound imaging equipment further comprises a mechanism for configuring the user interface in accordance with a user's personal preferences when the user logs in.

In an embodiment of the ultrasound imaging equipment of the present invention, the ultrasound imaging equipment further comprises a mechanism for configuring the user interface in accordance with an application selected by a user.

In an embodiment of the present invention, there is provided a method for automatically adjusting a layout of a user interface, comprising the steps of: powering on an ultrasound imaging equipment, the ultrasound imaging equipment comprising a host, a probe connected to the host, a touch screen fixed to the host for providing a user interface, the user interface including a presentation area displaying images and/or various information and a control area displaying virtual buttons; determining a relative position between the probe and the touch screen; and automatically adjusting a layout of the user interface based on the determined relative position, so as to facilitate a user's usage of the equipment.

In an embodiment of the method of the present invention, the method further comprises: prior to the automatic adjustment to a layout of the user interface, determining a state of the ultrasound imaging equipment, and enabling or disabling the automatic adjustment based on the determined state.

In an embodiment of the method of the present invention, the automatic adjustment is disabled when the ultrasound imaging equipment is in freeze; the automatic adjustment is disabled when the ultrasound imaging equipment is in a scan mode and the probe is in air; and the automatic adjustment is enabled when the ultrasound imaging equipment is in a scan mode and the probe is not in air.

In an embodiment of the method of the present invention, the step of determining a relative position between the probe and the touch screen comprises: transmitting first and second signals by first and second signal transmitters provided on the left and right sides of the touch screen respectively; receiving the first and second signals by a signal receiver disposed within the probe; and determining first and second distances based on the first and second signals received by the probe and determining a relative position between the probe and the touch screen by comparing the first and second distances, by a relative position analyzer. The step of automatically adjusting a layout of the user interface based on the determined relative position includes automatically setting by a user interface setter a layout of the presentation area and the control area in the user interface according to the determined relative position.

In an embodiment of the method of the present invention, the method further comprises transmitting the received first and second signals to the relative position analyzer in a wired or wireless manner by the probe.

In an embodiment of the method of the present invention, the method further comprises transmitting the determined relative position as an analytic result to the user interface setter in a wired or wireless manner by the relative position analyzer.

In an embodiment of the method of the present invention, when the first distance is greater than the second distance, the relative position analyzer determines that the probe is located on the right side of the touch screen, and accordingly, the user interface setter sets the control area on the left side of the presentation area; when the first distance is less than the second distance, the relative position analyzer determines that the probe is located on the left side of the touch screen, and accordingly, the user interface setter sets the control area on the right side of the presentation area.

In an embodiment of the method of the present invention, when the absolute value of a difference between the first and second distances is less than a threshold value, the user interface setter is disabled to maintain the current user interface; when the absolute value of the difference between the first and second distances is greater than the threshold value, the user interface setter is enabled.

In an embodiment of the method of the present invention, the threshold value is 20 cm.

In an embodiment of the method of the present invention, the step of determining a relative position between the probe and the touch screen includes: transmitting signals by a signal transmitter disposed within the probe; receiving respectively first and second signals transmitted from the signal transmitter by first and second signal receivers provided on the left and right sides of the touch screen respectively; determining first and second distances based on the first and second signals and determining a relative position between the probe and the touch screen by comparing the first and second distances, by a relative position analyzer. The step of automatically adjusting a layout of the user interface based on the determined relative position includes automatically setting by a user interface setter a layout of the presentation area and the control area in the user interface according to the determined relative position.

In an embodiment of the method of the present invention, the method further comprises transferring the received first and second signals to the relative position analyzer located in the host by the first and second signal receivers.

In an embodiment of the method of the present invention, the method further comprises pressing a physical key provided on the host or a virtual key provided on the touch screen to change a layout of the user interface by the user.

In an embodiment of the method of the present invention, the method further comprises voicing “switch the user interface” from the user to an acoustic control mechanism provided on the host to change a layout of the user interface.

In an embodiment of the method of the present invention, the method further comprises configuring the user interface in accordance with a user's personal preferences when the user logs in.

In an embodiment of the method of the present invention, the method further comprises configuring the user interface in accordance with an application selected by the user.

An embodiment of the present invention improves user experience.

An embodiment of the present invention saves a user's time.

An embodiment of the present invention simplifies the workflow.

### BRIEF DESCRIPTION OF THE DRAWINGS

The other objects, advantages and novelties of the present invention will become apparent through a detailed description of the present invention with reference to the accompanying drawings, in which:

FIG. 1 is a block diagram illustrating a structure of an ultrasound imaging equipment in accordance with an embodiment of the present invention;

FIG. 2 is a block diagram illustrating a structure of an ultrasound imaging equipment in accordance with an embodiment of the present invention;

FIG. 3 is a schematic diagram illustrating an exemplary implementation of a user interface adjusting device in accordance with an embodiment of the present invention;

FIG. 4 is a flow chart showing a method for automatically adjusting a layout of the user interface in accordance with an embodiment of the present invention; and

FIG. 5 is a flow chart showing a method for automatically adjusting a layout of the user interface in accordance with an embodiment of the present invention.

### DETAILED DESCRIPTION

The present invention is described herein below in more detail with reference to certain embodiments and the drawings. In order to facilitate illustration rather than to be limiting, the present disclosure sets forth particulars regarding, for example, specific devices, structures, techniques and the like, so that persons skilled in the art can easily appreciate the present invention. However, it should be understood that the present invention can also be practiced in other embodiments without the particulars described herein.

Skilled persons in the art should also appreciate that each component of the claimed equipment and/or each step of the claimed method may be realized, entirely or partially, via hardware, software and/or firmware. The present invention is not limited to any specific combination of hardware and software.

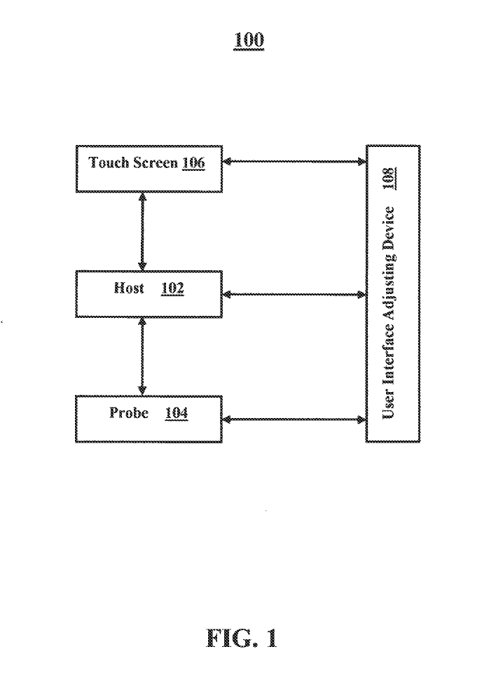

Referring now to FIG. 1, which schematically shows a structure of an ultrasound imaging equipment in accordance with an embodiment of the present invention. The ultrasound imaging equipment 100 comprises: a host 102 configured with various function modules for the completion of ultrasound imaging, such as a processor, memory, power supply, etc; a probe 104 connected to the host 102, for transmitting ultrasonic waves to scan a patient's body, receiving ultrasonic waves reflected back, converting received ultrasonic waves into an analog or digital signal and transmitting the same to the host 102 for further processing and analysis; in an embodiment of the present invention a touch screen 106 is fixed to the host for providing a user interface which can be roughly divided into a presentation area for displaying images (e.g., medical diagnostic images) and/or various information (e.g., text information, and menu), and a control area for displaying virtual buttons (or soft keys) to control and operate the ultrasound imaging equipment, including selection of certain parameters. The ultrasound imaging equipment 100 as shown further includes a user interface adjusting device 108 for automatically adjusting a layout of the user interface based on a relative position between the probe and the touch screen to facilitate usage of the equipment, such that, for example, the user can observe the presentation area while operating virtual buttons in the control area.

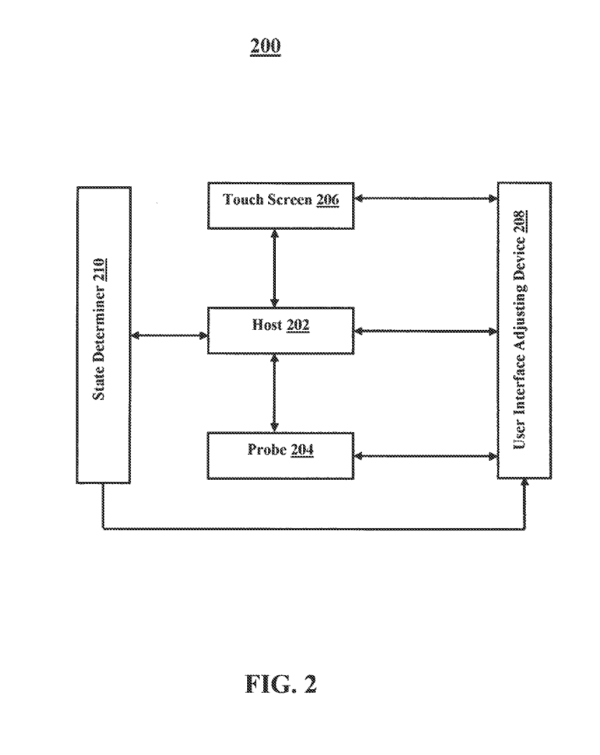

FIG. 2 is a block diagram illustrating a structure of an ultrasound imaging equipment in accordance with an embodiment of the present invention. The ultrasound imaging equipment 200 as shown comprises: a host 202; a probe 204 connected to the host; in an embodiment of the present invention a touch screen 206 is fixed to the host; and a user interface adjusting device 208. The host 202, the probe 204, the touch screen 206 and the user interface adjusting device 208 are substantially identical to the host 102, the probe 104, the touch screen 106 and the user interface adjusting device 108 of the equipment 100 illustrated in FIG. 1, and further elaborations thereupon are dispensed with herein.

The ultrasound imaging equipment 200 as shown further includes a state determiner 210, for determining a state of the ultrasound imaging equipment, and enabling or disabling the user interface adjusting device 208 based on the determined state.

States of the ultrasound imaging equipment include, for example, scan mode, freeze, and so on. The scan mode is a state where the current probe and associated ultrasound scanning hardware are in operation. Freeze refers to a state where the current probe and associated ultrasound scanning hardware are non-operative. In addition, when the user conducts non-scanning operations such as configuration, patient information manipulation and report completion, the system is also in freeze. In an embodiment, when the equipment is in the scan mode, the internal program will provide a status flag (for example, “Live”); when the equipment is in freeze, the internal program will provide another status flag (for example, “Freeze”). In an embodiment, the equipment is equipped with a button for switching between these two states. Besides, there is a special state referred to as “Probe in air”, which is a state under the scan mode. In this state, although the probe is in operation, it is in the air and does not contact the patient's body, such that all the images are noise signals. The internal program may also provide the state “Probe in air” with a corresponding status flag. Accordingly, the state determiner 210 may determine a state of the ultrasound imaging equipment based on the information provided by the internal program, thereby providing a control signal to enable or disable the user interface adjusting device 208. Of course, the state determiner 210 also may determine whether corresponding component(s) are in operation directly based on signals provided by the hardware in the equipment.

In an embodiment, when the ultrasound imaging equipment is in freeze, the user interface adjusting device 208 is disabled; when the ultrasound imaging equipment is in the scan mode and the probe is in air, the user interface adjusting device 208 is disabled; when the ultrasound imaging equipment is in the scan mode and the probe is not in air, the user interface adjusting device 208 is enabled.

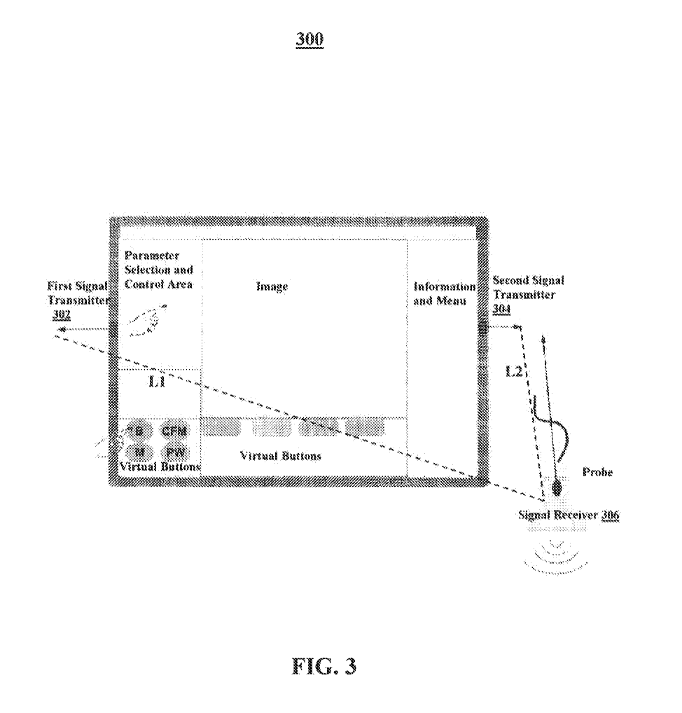

FIG. 3 is a schematic diagram illustrating an exemplary implementation of a user interface adjusting device in accordance with an embodiment of the present invention. The user interface adjusting device 300 includes: a first signal transmitter 302 and a second signal transmitter 304 provided on the left and right sides of a touch screen respectively, for transmitting a first signal and a second signal respectively (e.g., when the system is in the scan mode, the transmitters emit RF signals every 30 seconds); a signal receiver 306 provided within a probe, for receiving the first and second signals; a relative position analyzer (not shown), for determining a first distance L1 and a second distance L2 based on the first and second signals received by the probe, and for determining a relative position between the probe and touch screen by comparing the first distance L1 and the second distance L2; and a user interface setter (not shown) for automatically setting a layout of a presentation area and a control area in the user interface according to the determined relative position.

In an embodiment, both the relative position analyzer and the user interface setter are located within the host, for example, as part of the host, or in a same package shell together with the host. The probe transmits the received first and second signals to the relative position analyzer inside the host in a wired (e.g., via cables) or wireless (e.g., via Bluetooth) manner.

In an embodiment, the relative position analyzer is located within the probe, and is a separate member from the signal receiver 306. In an embodiment of the present invention, the relative position analyzer and the signal receiver 306 can be integrated to form a single piece; for example, some distance sensors or position sensors can simultaneously have the functionalities of a signal receiver and a relative position analyzer. The user interface setter is located within the host, and the relative position analyzer transmits the determined relative position as an analytic result to the user interface setter within the host in a wired or wireless manner.

In an embodiment, when the first distance L1 is greater than the second distance L2, the relative position analyzer determines that the probe is located on the right side of the touch screen, and accordingly, the user interface setter sets the control area on the left side of the presentation area, such that the right-sided presentation area will not be covered when the user operates the control area with his left hand. When the first distance L1 is less than the second distance L2, the relative position analyzer determines that the probe is located on the left side of the touch screen, and accordingly, the user interface setter sets the control area on the right side of the presentation area, so that the left-sided presentation area will not be covered when the user operates the control area with his right hand.

Of course, the user interface setter may, according to circumstances, place a part or the whole of the control area at the bottom of the screen, and place a part or the whole of the presentation area at the top of the screen. There may be other layouts for the presentation area and the control area, depending on actual design need.

In an embodiment, a threshold value (e.g., 20 cm, or a value between 10-30 cm) is provided in order to prevent from frequent changes of the interface layout when the two distances L1 and L2 show little difference. For example, when the absolute value of a difference between the first distance L1 and the second distance L2 is less than the threshold value, the user interface setter is disabled to maintain the current user interface; when the absolute value of the difference between the first distance L1 and the second distance L2 is greater than the threshold value, the user interface setter is enabled, and hence, interface layouts are dependent upon the magnitude of L1 and L2.

In an embodiment, the user interface adjusting device, different from the user interface adjusting device 300 as shown in FIG. 3, the signal transmitter is provided inside the probe for transmitting signals, and the first and second signal receivers are disposed on the left and right sides of the touch screen respectively, for receiving the first and second signals from the signal transmitter respectively.

In an embodiment, the relative position analyzer and the user interface setter are preferably located within the host, such that the first and second signal receivers on the touch screen can be easily in direct connection with the relative position analyzer within the host so as to transfer the received first and second signals to the relative position analyzer.

In an embodiment of the present invention, the ultrasound imaging equipment may also include a physical key on the host or a virtual key on the touch screen. When pressing either of these keys by the user, the layout of the user interface changes.

In an embodiment of the present invention, the ultrasound imaging equipment may also include an acoustic control device provided on the host, whereby when the user voices “switch the user interface”, the layout of the user interface changes.

In an embodiment of the present invention, the ultrasound imaging equipment may also include a device for identifying a user based on an inputted user name and the like when the user logs in, and then configuring the user interface in accordance with the user's personal preferences.

In an embodiment of the present invention, the ultrasound imaging equipment may also include a device for configuring the user interface in accordance with an application selected by a user. Doctors usually use one application to scan one anatomy. When scanning, doctors will keep one same pose and position relative to the equipment and the patient. It is therefore advantageous to configure different user interface layouts according to different applications.

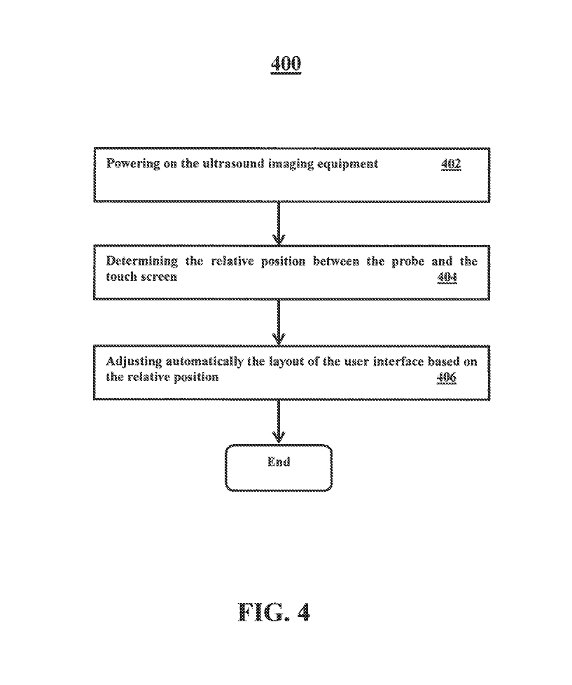

FIG. 4 is a flow chart showing a method for automatically adjusting a layout of the user interface in accordance with an embodiment of the present invention. The method 400 starts with step 402, where the ultrasound imaging equipment is powered on. At step 404, a relative position between the probe and the touch screen is determined. At step 406, a layout of the user interface is automatically adjusted based on the determined relative position.

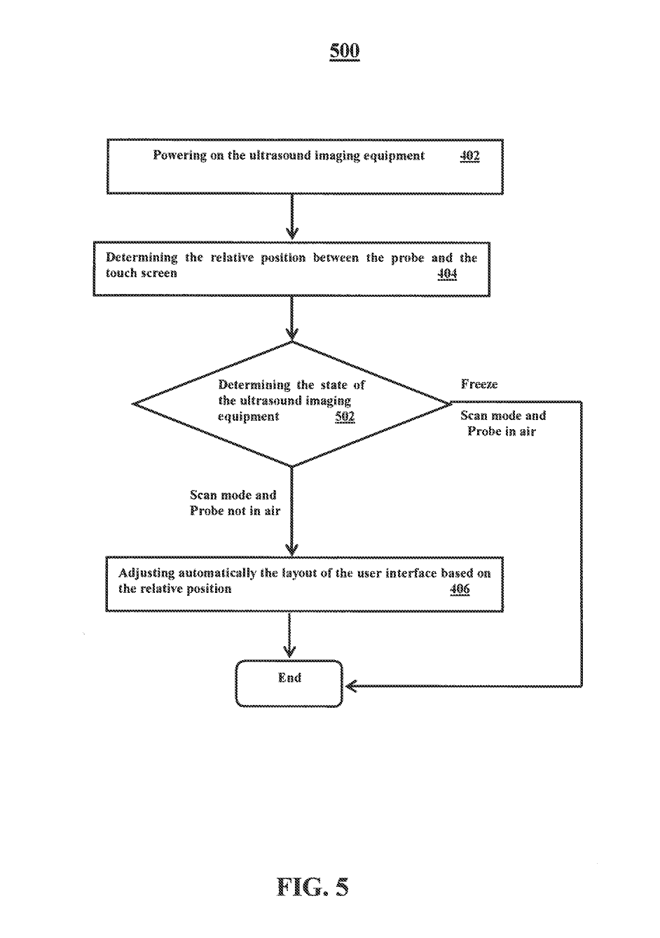

FIG. 5 is a flow chart showing a method for automatically adjusting a layout of the user interface in accordance with an embodiment of the present invention. The method 500 differs from the method 400 above in that, before automatically adjusting a layout of the user interface (step 406), a state of the ultrasound imaging equipment is determined (step 502), and then, the automatic adjustment step 406 is enabled or disabled based on the determined state.

In an embodiment, when the ultrasound imaging equipment is in freeze, the automatic adjustment is disabled; when ultrasound imaging equipment is in scan mode but the probe is in air, the automatic adjustment is disabled; when the ultrasound imaging equipment is in scan mode and the probe is not in air, the automatic adjustment is enabled.

In an embodiment, the step of determining a relative position may include: transmitting first and second signals by first and second signal transmitters provided on the left and right sides of the touch screen respectively; receiving the first and second signals by a signal receiver disposed within the probe; and determining first and second distances based on the first and second signals received by the probe and determining a relative position between the probe and the touch screen by comparing the first and second distances, by a relative position analyzer. The step of automatically adjusting a layout of the user interface includes automatically setting by a user interface setter a layout of the presentation area and the control area in the user interface according to the determined relative position.

The method further may comprise transmitting the received first and second signals to the relative position analyzer in a wired or wireless manner by the probe.

The method further may comprise transmitting the determined relative position as an analytic result to the user interface setter in a wired or wireless manner by the relative position analyzer.

In an embodiment of the method, when the first distance is greater than the second distance, it is determined that the probe is located on the right side of the touch screen, and accordingly, the control area is set on the left side of the presentation area; when the first distance is less than the second distance, it is determined that the probe is located on the left side of the touch screen, and accordingly, the control area is set on the right side of the presentation area.

In addition, a threshold value (e.g., a value between 10-30 cm, such as 20 cm) may be set. When the absolute value of a difference between the first distance and the second distance is less than the threshold value, the user interface setter is disabled to maintain the current user interface; when the absolute value of the difference between the first and second distances is greater than the threshold value, the user interface setter is enabled.

In an embodiment, the step of determining a relative position can be modified as follows: transmitting signals from a signal transmitter disposed in the probe; and receiving first and second signals from the signal transmitter by first and second signal receivers respectively that are provided on the left and right sides of the touch screen respectively.

The method may also include transferring the received first and second signals to the relative position analyzer located within the host by the first and second signal receivers.

In an embodiment, the method may also include pressing by the user a physical key provided on the host or a virtual key provided on the touch screen to change a layout of the user interface. In an embodiment of the present invention, the method may also comprise voicing “switch the user interface” from the user to an acoustic control mechanism provided on the host to change a layout of the user interface. In an embodiment of the present invention, the method may also comprise configuring the user interface in accordance with a user's personal preferences when the user logs in. In an embodiment of the present invention, the method may also comprise configuring the user interface in accordance with an application elected by a user. One or more of these modes may be used in combination.

The automatic adjustment mode according to embodiments of the present invention can be used in combination with other adjusting manners. Embodiments of the present invention may apply to various devices equipped with a touch screen, so as to improve the user experience, save the user's time, and simplify the workflow.

It should be noted that the phases “one embodiment”, “another embodiment” and the like used herein do not necessarily refer to same or different embodiments. In other words, the skilled in the art can understand that the technical features or technical solutions of those embodiments described herein can be used in combination or separately according to circumstances.

Embodiments of the present invention described herein are illustrated by way of example and not by way of limitation. Although specific terms may be adopted herein, they are only used in a general and descriptive sense, and are not for purposes of limitation. For example, the term equipment shall be interpreted in a broad sense to mean a piece of equipment, station, device, machine, system, apparatus, appliance, etc. that is portable and/or stationary. The scope of the present invention is defined only by the appended claims and equivalents thereof

What is claimed is:

1. An ultrasound imaging equipment, comprising:
a host;
a probe connected to the host;
a touch screen fixed to the host and configured to provide a user interface, the user interface comprising a presentation area configured to display images and/or various information and a control area configured to display virtual buttons; and
a user interface adjusting device configured to automatically adjust a layout of the user interface based on a relative position between the probe and the touch screen, so as to facilitate a usage of the ultrasound imaging equipment.

2. The ultrasound imaging equipment according to claim 1, further comprising:
a state determiner configured to determine a state of the ultrasound imaging equipment, and to enable or disable the user interface adjusting device based on the determined state.

3. The ultrasound imaging equipment according to claim 2, wherein:
the user interface adjusting device is disabled when the ultrasound imaging equipment is in freeze,
the user interface adjusting device is disabled when the ultrasound imaging equipment is in a scan mode and the probe is in air, and
the user interface adjusting device is enabled when the ultrasound imaging equipment is in a scan mode and the probe is not in air.

4. The ultrasound imaging equipment according to claim 1, wherein the user interface adjusting device comprises:
a first signal transmitter on the left side of the touch screen, wherein the first signal transmitter is configured to transmit a first signal;
a second signal transmitter on the right side of the touch screen, wherein the second signal transmitter is configured to transmit a second signal;
a signal receiver disposed within the probe and configured to receive the first signal and the second signal;
a relative position analyzer configured to determine a first distance and a second distance based on the first signal and the second signal received by the probe, and to determine a relative position between the probe and the touch screen by comparing the first distance and the second distance; and
a user interface setter configured to automatically set a layout of the presentation area and the control area in the user interface according to the determined relative position.

5. The ultrasound imaging equipment according to claim 4, wherein the relative position analyzer and the user interface setter are both located within the host, and the probe transmits the received first signal and the received second signal to the relative position analyzer in a wired or wireless manner.

6. The ultrasound imaging equipment according to claim 4, wherein the relative position analyzer is located within the probe, the user interface setter is located within the host, and the relative position analyzer transmits the determined relative position as an analytic result to the user interface setter in a wired or wireless manner.

7. The ultrasound imaging equipment according to claim 4, wherein:
when the first distance is greater than the second distance, the relative position analyzer determines that the probe is located on the right side of the touch screen, and accordingly, the user interface setter sets the control area on the left side of the presentation area,
when the first distance is less than the second distance, the relative position analyzer determines that the probe is located on the left side of the touch screen, and accordingly, the user interface setter sets the control area on the right side of the presentation area.

8. The ultrasound imaging equipment according to claim 7, wherein:
when an absolute value of a difference between the first distance and the second distance is less than a threshold value, the user interface setter is disabled to maintain the current user interface, and
when the absolute value of the difference between the first distance and the second distance is greater than the threshold value, the user interface setter is enabled.

9. The ultrasound imaging equipment according to claim 8, wherein the threshold value is about 20 cm.

10. The ultrasound imaging equipment according to claim 1, wherein the user interface adjusting device comprises:
a signal transmitter disposed in the probe, and configured to transmit a first signal and a second signal;
a first signal receiver on the left side of the touch screen configured to receive the first signal from the signal transmitter;
a second signal receiver on the right side of the touch screen configured to receive the second signal from the signal transmitter;
a relative position analyzer configured to determine a first distance and a second distance based on the first signal and the second signal and to determine a relative position between the probe and the touch screen by comparing the first distance and the second distance; and
a user interface setter configured to automatically set a layout of the presentation area and the control area in the user interface according to the determined relative position.

11. The ultrasound imaging equipment according to claim 10, wherein the relative position analyzer and the user interface setter are located within the host, and the first signal receiver and the second signal receiver are connected to the relative position analyzer so as to transfer the received first signal and the received second signal to the relative position analyzer.

12. The ultrasound imaging equipment according to claim 1, further comprising a physical key provided on the host or a virtual key provided on the touch screen, upon pressing the physical key or the virtual key the layout of the user interface changes.

13. The ultrasound imaging equipment according to claim 1, further comprising an acoustic control mechanism provided on the host, whereby when the user roices a command, the layout of the user interface changes.

14. The ultrasound imaging equipment according to claim 1, further comprising a user interface adjusting device configured to configure the user interface in accordance with the user's personal preferences when the user logs in.

15. The ultrasound imaging equipment according to claim 14, further comprising a user interface adjusting device configured to configure the user interface in accordance with an application selected by the user.

16. A method for automatically adjusting a layout of a user interface, the method comprising:
powering on an ultrasound imaging equipment, the ultrasound imaging equipment comprising a host, a probe connected to the host, a touch screen fixed to the host and configured to provide a user interface, the user interface comprising a presentation area configured to display images and/or various information and a control area configured to display virtual buttons;
determining a relative position between the probe and the touch screen; and
automatically adjusting the layout of the user interface based on the determined relative position, so as to facilitate a user's usage of the ultrasound imaging equipment.

17. The method according to claim 16, wherein prior to automatically adjusting the layout of the user interface, the method further comprising:
determining a state of the ultrasound imaging equipment; and
enabling or disabling the automatic adjustment based on the determined state.

18. The method according to claim 17, further comprising:
disabling the automatic adjustment of the layout of the user interface when the ultrasound imaging equipment is in freeze;
disabling the automatic adjustment of the layout of the user interface when the ultrasound imaging equipment is in a scan mode and the probe is in air; and
enabling the automatic adjustment of the layout of the user interface when the ultrasound imaging equipment is in a scan mode and the probe is not in air.

19. The method according to claim 16, wherein determining a relative position between the probe and the touch screen comprises:
transmitting a first signal via a first signal transmitter on the left side of the touch screen;
transmitting a second signal via a second signal transmitter on the right side of the touch screen;
receiving the first signal and the second signal via a signal receiver disposed within the probe; and
determining a first distance and a second distance based on the first signal and the second signal received by the probe and determining a relative position between the probe and the touch screen by comparing the first distance and the second distance, by a relative position analyzer,
wherein adjusting automatically a layout of the user interface comprises setting automatically by a user interface setter a layout of the presentation area and the control area in the user interface according to the determined relative position.

20. The method according to claim 19, further comprising:
transmitting by the probe the received first signal and the received second signal to the relative position analyzer in a wired or a wireless manner.

21. The method according to claim 19, further comprising:
transmitting by the relative position analyzer the determined relative position as an analytic result to the user interface setter in a wired or a wireless manner.

22. The method according to claim 19, wherein:
when the first distance is greater than the second distance, the relative position analyzer determines that the probe is located on the right side of the touch screen, and accordingly, the user interface setter sets the control area on the left side of the presentation area, and
when the first distance is less than the second distance, the relative position analyzer determines that the probe is located on the left side of the touch screen, and accordingly, the user interface setter sets the control area on the right side of the presentation area.

23. The method according to claim 22, wherein:
when an absolute value of a difference between the first distance and the second distance is less than a threshold value, the user interface setter is disabled to maintain the current user interface, and
when the absolute value of the difference between the first distance and the second distance is greater than the threshold value, the user interface setter is enabled.

24. The method according to claim 23, wherein the threshold value is about 20 cm.

25. The method according to claim 16, wherein determining a relative position between the probe and the touch screen comprises:
transmitting signals by a signal transmitter disposed within the probe;
receiving a first signal of the signals transmitted from the signal transmitter by a first signal receiver on the left side of the touch screen;
receiving a second signal transmitted from the signal transmitter by a second signal receiver on the right side of the touch screen;
determining a first distance and a second distance based on the first signal and the second signal and determining the relative position between the probe and the touch screen by comparing the first distance and the second distance, by a relative position analyzer, wherein adjusting automatically the layout of the user interface based on the determined relative position comprises:
setting automatically by a user interface setter a layout of the presentation area and the control area in the user interface according to the determined relative position.

26. The method according to claim 25, further comprising:
transferring the received first signal and the received second signal to the relative position analyzer located in the host by the first signal receiver and the second signal receiver.

27. The method according to claim 16, further comprising:
pressing by the user a physical key provided on the host or a virtual key provided on the touch screen to change the layout of the user interface.

28. The method according to claim 16, further comprising:
voicing a command from the user to an acoustic control mechanism on the host to change the layout of the user interface.

29. The method according to claim 16, further comprising:
configuring the user interface in accordance with the user's personal preferences when the user logs in.

30. The method according to claim 29, further comprising:
configuring the user interface in accordance with an application selected by the user.
